(19) United States
(12) Patent Application Publication
(10) Pub. No.: US 2025/0277246 A1
(43) Pub. Date: Sep. 4, 2025

(54) EFFICIENT ENZYMATIC SYNTHESIS METHOD FOR HOLOTHURIAN GLYCOSAMINOGLYCAN

(71) Applicant: Institute of Oceanology, Chinese Academy of Sciences, Qingdao Shandong (CN)

(72) Inventors: Rongfeng LI, Qingdao Shandong (CN); Yuanjie LIU, Qingdao Shandong (CN); Kaiqiang LI, Qingdao Shandong (CN); Huahua YU, Qingdao Shandong (CN); Rong’e XING, Qingdao Shandong (CN); Song LIU, Qingdao Shandong (CN); Pengcheng LI, Qingdao Shandong (CN)

(21) Appl. No.: 19/211,103

(22) Filed: May 16, 2025

(30) Foreign Application Priority Data
Aug. 16, 2024 (CN) 202411132030.8

(51) Int. Cl. C12P 19/04; C12N 9/10; C12N 9/12

(52) U.S. Cl. CPC C12P 19/04; C12N 9/1051; C12N 9/1205; C12Y 204/01065; C12Y 207/01052; C12Y 207/0703

(57) ABSTRACT

The present invention belongs to the technical field of biochemistry, and in particular relates to an efficient enzymatic synthesis method for a holothurian glycosaminoglycan. The present invention establishes an efficient enzymatic synthesis route for the holothurian glycosaminoglycan, in which the holothurian glycosaminoglycan with anticoagulant activity is efficiently synthesized through a one-pot multienzymatic strategy using sulfated fucose and chondroitin sulfate as substrates and catalyzed by α-1,3-fucosyltransferase mutant and L-fucokinase/GDP-fucose pyrophosphorylase, which provides important technical support for the development of new anticoagulant drugs.

### TECHNICAL FIELD

The present invention belongs to the technical field of biochemistry, and in particular relates to an efficient enzymatic synthesis method for the holothurian glycosaminoglycan.

### BACKGROUND

Holothurian glycosaminoglycan, also known as Fucosylated Chondroitin Sulfate (FCS), is a high molecular weight mucopolysaccharide composed of chondroitin sulfate as a main chain and sulfated fucose as a branch chain, and has significant anticoagulant, antioxidant, anti-inflammatory, anti-tumor and other important biological activities. Among them, the anticoagulant activity is the most eye-catching, and is stronger than clinical heparin drugs. Moreover, holothurian glycosaminoglycan acts on the intrinsic anticoagulant pathway, and has an extremely low risk of bleeding and other side effects. Therefore, holothurian glycosaminoglycan is a promising candidate for developing new, safe, and efficient anticoagulant drugs. However, at present, holothurian glycosaminoglycan is mainly extracted from natural sea cucumber by multiple extraction operations and separation steps. In addition, naturally obtained holothurian glycosaminoglycan is a mixture composed of many different sulfation patterns with heterogeneous structures, so it is impossible to obtain a holothurian glycosaminoglycan with a single structure by the traditional separation method. Moreover, glycosaminoglycans extracted from different species of sea cucumber have very different structures and compositions, which poses certain difficulties to the development of new anticoagulant drugs. Artificial synthesis is an effective method to obtain a holothurian glycosaminoglycan with a uniform structure. Currently, a 12-step chemical synthesis route has been developed for the production of low molecular weight holothurian glycosaminoglycan (nonasaccharide). However, this method requires multiple protection and deprotection steps, making the process cumbersome and resulting in a low overall yield of only 3%. Moreover, to date, no synthetic methodology has been established for the chemical synthesis of high molecular weight holothurian glycosaminoglycans polysaccharides.

### SUMMARY

An object of the present invention is to solve the problems in the prior art and provide an efficient enzymatic synthesis route for the holothurian glycosaminoglycan, in which the holothurian glycosaminoglycan with excellent anticoagulant activity is efficiently synthesized through a one-pot multienzymatic strategy using sulfated fucose and chondroitin sulfate as substrates and catalyzed by α-1,3-fucosyltransferase and a mutant thereof and L-Fucokinase/L-Fucose-1-P guanylyltransferase (FKP), which provides important technical support for the development of new FCS anticoagulant drugs.

In order to achieve the above object, the present invention adopts the following technical solution: an efficient enzymatic synthesis method for a holothurian glycosaminoglycan, where sulfated fucose and chondroitin sulfate are used as substrates to prepare the holothurian glycosaminoglycan with anticoagulant activity by one-pot multi-enzymatic synthesis strategy using α-1,3-fucosyltransferase (α1,3FucT, GenBank: AAD07447.1) mutant and L-fucokinase/Guanosine Diphosphate (GDP)-Fucose Pyrophosphorylase (FKP, GenBank: AAX45030.1) in the presence of Adenosine Triphosphate (ATP) and Guanosine Triphosphate (GTP); a molar ratio of the chondroitin sulfate to the sulfated fucose is 1:100-1:1200; a molar ratio of the chondroitin sulfate to the α-1,3-fucosyltransferase mutant is 1:1-3:1; a molar ratio of the sulfated fucose to the FKP is 25:1-75:1; a molar ratio of the sulfated fucose to the ATP and the GTP is 1:1-1:3; and the α-1,3-fucosyltransferase mutant deletes 52 amino acid residues at a C-terminus of the protease, and mutates serine at position 46 to phenylalanine, glycine at position 128 to asparagine, histidine at position 129 to glutamate and tyrosine at position 132 to isoleucine.

Preferably, the chondroitin sulfate is chondroitin sulfate A.

Preferably, a concentration of the sulfated fucose is 1-5 mM.

Preferably, a reaction time is 12-96 h.

Preferably, a reaction pH is 7.0-8.0.

Preferably, a reaction temperature is 25-35° C.

Preferably, 10-30 mM MgCl₂ is added to the reaction.

Compared with the prior art, the present invention has the following advantages: (1) the glycosaminoglycan synthesized by the enzymatic method of the present invention has a uniform structure and is completely the same as the natural holothurian glycosaminoglycan; (2) the present invention only requires one reaction step, has simple operation, high efficiency, and high yield; and (3) the synthesized holothurian glycosaminoglycan of the present invention has strong intrinsic anticoagulant activity and is expected to be developed into a new safe and efficient anticoagulant drug.

### BRIEF DESCRIPTION OF THE DRAWINGS

FIG. 1 is a schematic diagram of an efficient enzymatic synthesis method for the holothurian glycosaminoglycan according to the present invention;

FIG. 2 is a molecular weight determination diagram of the holothurian glycosaminoglycan in Embodiment 3 and Embodiment 4 of the present invention by High-Performance Liquid Chromatography (HPLC);

FIG. 3 is the Nuclear Magnetic Resonance Hydrogen Spectrum (1H-NMR) diagram of the holothurian glycosaminoglycan in Embodiment 3 of the present invention;

FIG. 4 is the Nuclear Magnetic Resonance Carbon Spectrum (¹³C-NMR) diagram of the holothurian glycosaminoglycan in Embodiment 3 of the present invention;

FIG. 5 is the Nuclear Magnetic Resonance Overhauser effect (NOE) spectrogram of the holothurian glycosaminoglycan in Embodiment 3 of the present invention;

FIG. 6 is the Nuclear Magnetic Resonance Hydrogen Spectrum (1H-NMR) diagram of the holothurian glycosaminoglycan in Embodiment 4 of the present invention;

FIG. 7 is the Nuclear Magnetic Resonance Carbon Spectrum (¹³C-NMR) diagram of the holothurian glycosaminoglycan in Embodiment 4 of the present invention;

FIG. 8 is the Nuclear Magnetic Resonance Overhauser effect (NOE) spectrogram of the holothurian glycosaminoglycan in Embodiment 4 of the present invention; and

FIG. 9 is a graph of intrinsic anticoagulant APTT activity of holothurian glycosaminoglycans in Embodiment 3 and Embodiment 4 of the present invention.

### DESCRIPTION OF THE EMBODIMENTS

In order to facilitate understanding of the present invention, the present invention will be described in more detail below with reference to the accompanying drawings and specific embodiments. Preferred embodiments of the present invention are shown in the drawings. The present invention may, however, be embodied in many different forms, and should not be construed as limited to the embodiments set forth herein. Rather, these embodiments are provided to enable the disclosure to be understood thoroughly and completely.

The α-1,3-fucosyltransferase mutant used in the embodiments of the present invention is a mutant obtained by deleting 52 amino acid residues at a C-terminus of the protease, and mutating serine at position 46 to phenylalanine, glycine at position 128 to asparagine, histidine at position 129 to glutamate and tyrosine at position 132 to isoleucine (α1,3FucT-S46F/A128N/H129E/Y132I/ΔC52), as described in Yun Hee Choi et al. Solubilization and Iterative Saturation Mutagenesis of a1,3-Fucosyltransferase From Helicobacter pylori to Enhance Its Catalytic Efficiency [J]. Biotechnology and Bioengineering, vol. 9999, No. xxx, 2016.

### Embodiment 1

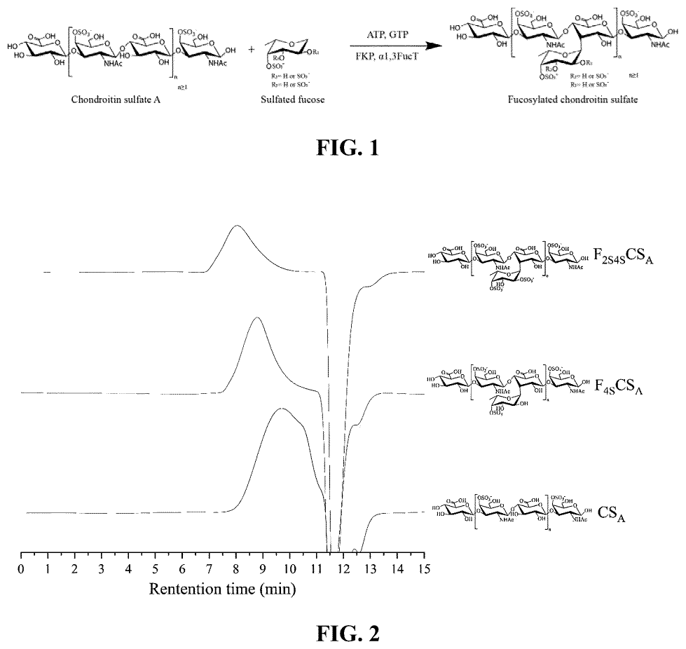

The principle of the enzymatic synthesis reaction of the holothurian glycosaminoglycan provided in the present embodiment is shown in FIG. 1, and the steps are as follows.

Totally 1 mM 4-O-sulfated fucose, 1 mM ATP, 1 mM GTP, 10 μM chondroitin sulfate A (molecular weight of 1 kDa), 10 mM MgCl₂ were placed in 10 mM Tris-HCl at pH 7.0, to which 3.3 mM α-1,3-fucosyltransferase mutant and 14 μM FKP were added, and they were oscillated at 25° C. for 12 h for reaction. After the reaction was finished, the enzyme was inactivated by boiling, and a supernatant was obtained by centrifugation. The supernatant was dialyzed in a 500 Da dialysis bag to obtain a dialyzate, and the dialyzate was concentrated and freeze-dried to obtain the product holothurian glycosaminoglycan.

### Embodiment 2

The principle of the enzymatic synthesis reaction of the holothurian glycosaminoglycan provided in the present embodiment is shown in FIG. 1, and the steps are as follows.

Totally 2.5 mM 4-O-sulfated fucose, 5 mM ATP, 5 mM GTP, 3 μM chondroitin sulfate A (molecular weight of 30 kDa), 20 mM MgCl₂ were placed in 10 mM Tris-HCl at pH 7.5, to which 1.5 μM α-1,3-fucosyltransferase mutant and 50 μM FKP were added, and they were oscillated at 30° C. for 48 h for reaction. After the reaction was finished, the enzyme was inactivated by boiling, and a supernatant was obtained by centrifugation. The supernatant was dialyzed in an 8000-14000 Da dialysis bag to obtain a dialyzate, and the dialyzate was concentrated and freeze-dried to obtain the product holothurian glycosaminoglycan.

### Embodiment 3

The principle of the enzymatic synthesis reaction of the holothurian glycosaminoglycan provided in the present embodiment is shown in FIG. 1, and the steps are as follows.

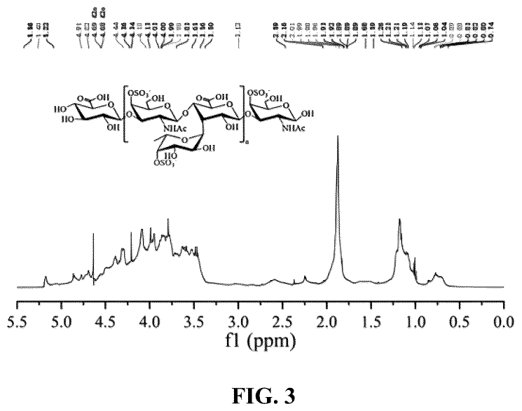

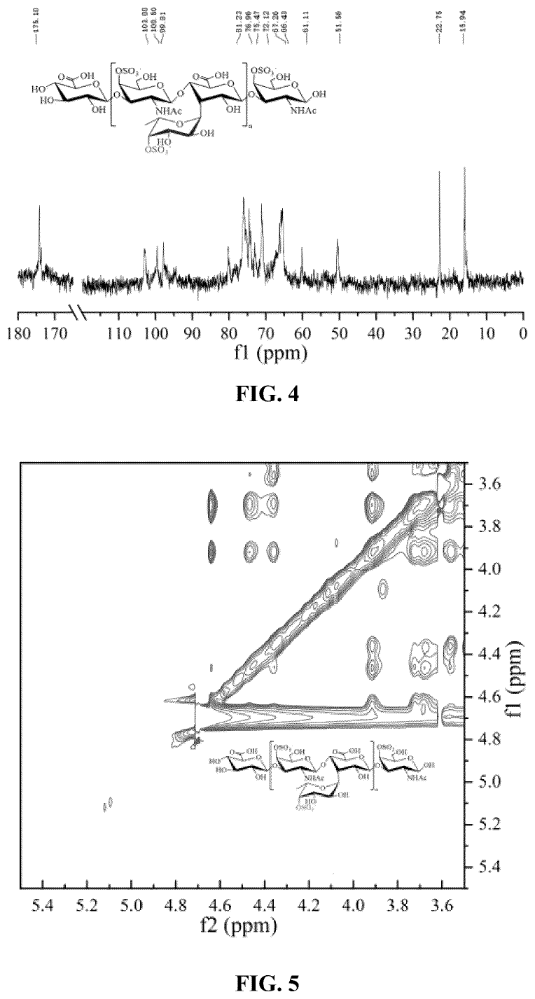

Totally 5 mM 4-O-sulfated fucose, 15 mM ATP, 15 mM GTP, 4.2 μM chondroitin sulfate A (molecular weight of 35 kDa), 20 mM MgCl₂ were placed in 10 mM Tris-HCl at pH 8.0, to which 4.2 μM α-1,3-fucosyltransferase mutant and 0.2 mM FKP were added, and they were oscillated at 35° C. for 96 h for reaction. After the reaction was finished, the enzyme was inactivated by boiling, and a supernatant was obtained by centrifugation. The supernatant was dialyzed in an 8000-14000 Da dialysis bag to obtain a dialyzate, and the dialyzate was concentrated and freeze-dried to obtain the product holothurian glycosaminoglycan F_{4S}CS_{A} with a yield of 63.7%. The synthesized product was subject to High-Performance Liquid Chromatography (HPLC) for molecular weight determination, as well as Nuclear Magnetic Resonance Hydrogen Spectrum (¹H-NMR), Nuclear Magnetic Resonance Carbon Spectrum (¹³C-NMR) and Nuclear Magnetic Resonance Overhauser Effect (NOE) spectrogram for structural identification and analysis, and the determination results are shown in FIGS. 2, 3, 4, and 5 respectively. The results show that the holothurian glycosaminoglycan synthesized by the method of the present invention is completely the same as the natural holothurian glycosaminoglycan.

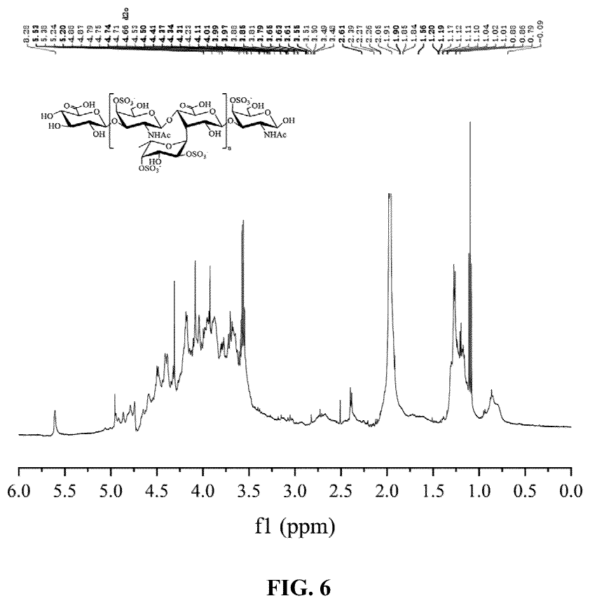

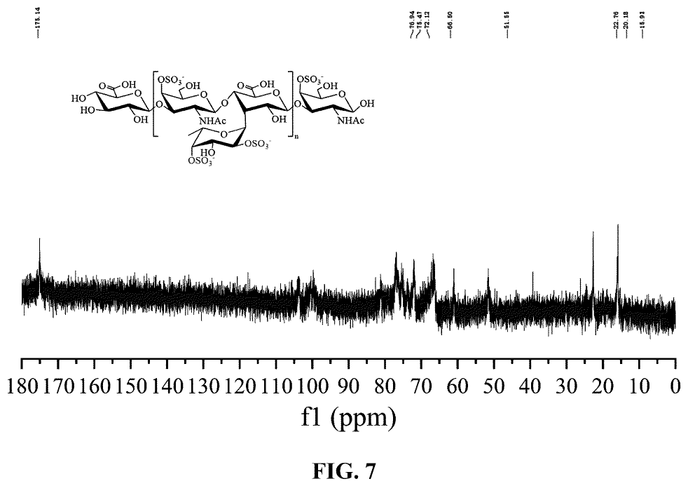

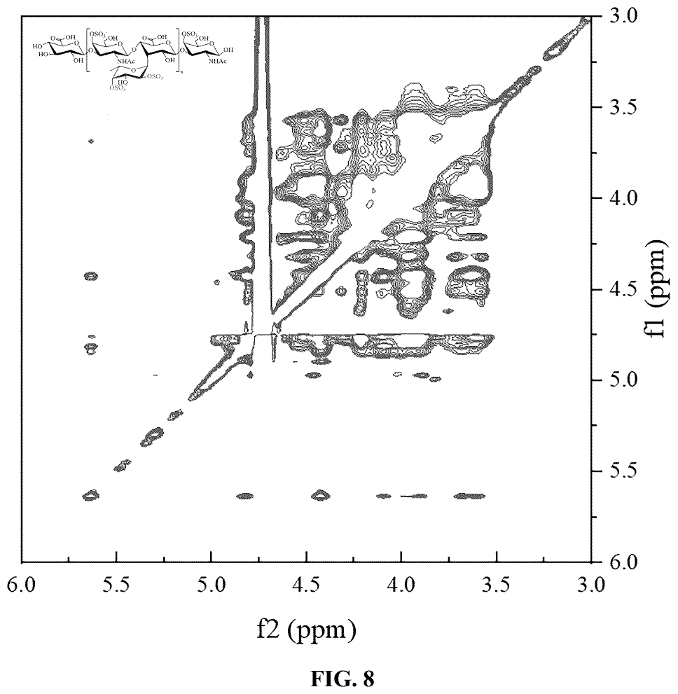

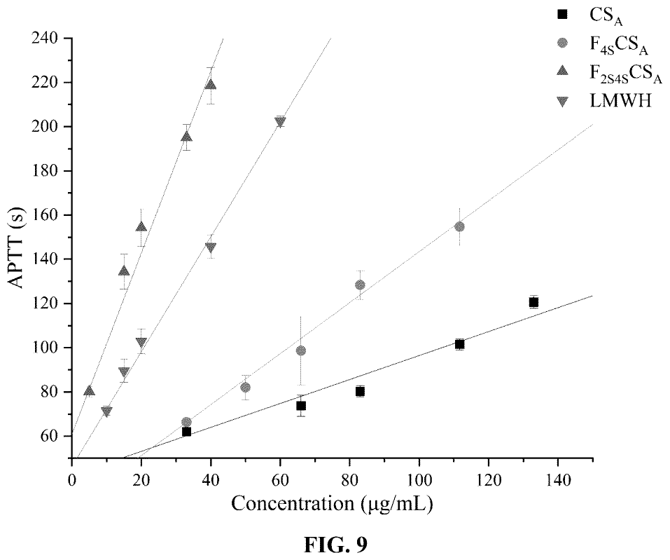

The test method for the intrinsic anticoagulant activity (APTT) of the synthesized holothurian glycosaminoglycan F_{4S}CS_{A} is as follows: total 10 μL sample solutions of different concentrations, Low Molecular Weight Heparin (LMWH) as a positive control, Chondroitin Sulfate A (CS_{A}) and PBS isotonic buffer as negative controls were respectively added to test tubes preheated at 37° C., to which 30 μL sheep plasma and 30 μL APTT reagent were added and incubated at 37° C. for 5 min, and then 30 μL 0.025 M CaCl₂ solution preheated at 37° C. was quickly added to the test tubes while starting timing, and coagulation time was recorded. The test results are shown in FIG. 6. The holothurian glycosaminoglycan F_{4S}CS_{A} synthesized by the method of the present invention has excellent intrinsic anticoagulant activity, which provides important technical support for the development of new anticoagulant drugs.

### Embodiment 4

The enzymatic synthesis reaction steps of the holothurian glycosaminoglycan provided in the present embodiment are as follows.

Totally 5 mM 2,4-O-disulfated fucose, 15 mM ATP, 15 mM GTP, 4.2 μM chondroitin sulfate A (molecular weight of 35 kDa), 20 mM MgCl₂ were placed in 10 mM Tris-HCl at pH 8.0, to which 4.2 μM α-1,3-fucosyltransferase mutant and 0.2 mM FKP was added, and they oscillated at 35° C. for 96 h for reaction. After the reaction was finished, the enzyme was inactivated by boiling, and a supernatant was obtained by centrifugation. The supernatant was dialyzed in an 8000-14000 Da dialysis bag to obtain a dialyzate, and the dialyzate was concentrated and freeze-dried to obtain the product holothurian glycosaminoglycan F_{2S4S}CS_{A} with a yield of 60.8%. The synthesized product was subject to High-Performance Liquid Chromatography (HPLC) for molecular weight determination, as well as Nuclear Magnetic Resonance Hydrogen Spectrum (¹H-NMR), Nuclear Magnetic Resonance Carbon Spectrum (¹³C-NMR) and Nuclear Magnetic Resonance Overhauser Effect (NOE) spectrogram for structural identification and analysis.

The test method for the intrinsic anticoagulant activity (APTT) of the synthesized holothurian glycosaminoglycan F_{2S4S}CS_{A} is as follows: total 10 μL sample solutions of different concentrations, Low Molecular Weight Heparin (LMWH) as a positive control, Chondroitin Sulfate A (CS_{A}) and PBS isotonic buffer as negative controls were respectively added to test tubes preheated at 37° C., to which 30 μL sheep plasma and 30 μL APTT reagent were added and incubated at 37° C. for 5 min, and then 30 μL 0.025 M CaCl₂ solution preheated at 37° C. was quickly added to the test tubes while starting timing, and coagulation time was recorded.

What is claimed is:

1. An efficient enzymatic synthesis method for a holothurian glycosaminoglycan, wherein sulfated fucose and chondroitin sulfate are used as substrates to prepare the holothurian glycosaminoglycan with anticoagulant activity by one-pot multi-enzymatic synthesis strategy using α-1,3-fucosyltransferase mutant and L-fucokinase/GDP-fucose pyrophosphorylase in the presence of ATP and GTP; a molar ratio of the chondroitin sulfate to the sulfated fucose is 1:(100-1200); a molar ratio of the chondroitin sulfate to the α-1,3-fucosyltransferase mutant is (1-3):1; a molar ratio of the sulfated fucose to the L-fucokinase/GDP-fucose pyrophosphorylase is (25-75):1; a molar ratio of the sulfated fucose to the ATP and the GTP is 1:(1-3); and the α-1,3-fucosyltransferase mutant deletes 52 amino acid residues at a C-terminus of the protease, and mutates serine at position 46 to phenylalanine, glycine at position 128 to asparagine, histidine at position 129 to glutamate and tyrosine at position 132 to isoleucine.

2. The efficient enzymatic synthesis method for the holothurian glycosaminoglycan according to claim 1, wherein the chondroitin sulfate is chondroitin sulfate A.

3. The efficient enzymatic synthesis method for the holothurian glycosaminoglycan according to claim 1, wherein a concentration of the sulfated fucose is 1-5 mM.

4. The efficient enzymatic synthesis method for the holothurian glycosaminoglycan according to claim 1, wherein a reaction time is 12-96 h.

5. The efficient enzymatic synthesis method for the holothurian glycosaminoglycan according to claim 1, wherein a reaction pH is 7.0-8.0.

6. The efficient enzymatic synthesis method for the holothurian glycosaminoglycan according to claim 1, wherein a reaction temperature is 25-35° C.

7. The efficient enzymatic synthesis method for the holothurian glycosaminoglycan according to claim 1, wherein 10-30 mM MgCl₂ is added to the reaction.
